(19) United States
(12) Patent Application Publication
(10) Pub. No.: US 2024/0122487 A1
(43) Pub. Date: Apr. 18, 2024

(54) METHOD OF DETERMINING SYSTOLIC AND DIASTOLIC BLOOD PRESSURE AND THE UNIT FOR THIS METHOD

(71) Applicant: CZECH TECHNICAL UNIVERZITY IN PRAGUE, FACULTY OF ELECTRICAL ENGINEERING, DEPARTMENT, Praha 6 (CZ)

(72) Inventors: Jiri CHOD, Praha (CZ); Pavel ZAHRADNIK, Praha (CZ)

(73) Assignee: CZECH TECHNICAL UNIVERZITY IN PRAGUE, FACULTY OF ELECTRICAL ENGINEERING, DEPARTMENT, Praha 6 (CZ)

(21) Appl. No.: 18/235,966

(22) Filed: Aug. 21, 2023

Related U.S. Application Data
(63) Continuation of application No. 16/543,831, filed on Aug. 19, 2019, now abandoned.
Application No. 16/543,831 is a continuation of application No. 14/898,640, filed on Dec. 15, 2015, now abandoned.

(30) Foreign Application Priority Data
Jun. 21, 2013 (CZ) PV 2013-478
Jun. 24, 2013 (CZ) PV2013-488
May 23, 2014 (CZ) PV 2014-356
Jun. 2, 2014 (CZ) PV 2014-375

(51) Int. Cl. A61B 5/021; A61B 5/00; A61B 5/026; A61B 5/0295; A61B 5/1455

(52) U.S. Cl. CPC A61B 5/02108; A61B 5/0024; A61B 5/02116; A61B 5/02141; A61B 5/0261; A61B 5/0295; A61B 5/14551; A61B 5/6802; A61B 5/6816; A61B 5/6826; A61B 5/7235; A61B 2560/0252

(57) ABSTRACT

The method of determining systolic and diastolic blood pressure where the set of received data of each curve of the plethysmographic curve gained from the unit for monitoring the life functions in a shape of the ring and/or the earring is divided into individual segments that respond to one heartbeat of the measured subject and after processing there are selected and recalculated segments meeting the defined criteria. The unit for monitoring and measuring the life functions comprises a supporting construction in a shape of a ring (29) and/or an earring (39).

### TECHNICAL FIELD

The invention concerns the method of determining systolic and diastolic blood pressure and the unit enabling to carry out this method. It is the technological solution for determining the systolic and diastolic blood pressure that enables monitoring and measuring humane life functions and utilizes the telecommunication technology with electronics and sensors, computer technology and the wireless communication, factually the solution concerns the unit for monitoring and measuring human life functions by a specifically adapted electronic module and its communication with other devices.

### BACKGROUND ART

Blood pressure is one of the most important physiologic parameters of a human organism. Its measuring belongs among routine procedures in medicine and is a part of most of the medical check-ups.

Considering the fact that around 50% adults out of adult population suffers from the high blood pressure (hypertension), or they are at the stage of prehypertension, there arises a need of frequent control of real values of the blood pressure, because the deviation from the optimal values can usually bring serious health problems, oftentimes fatal. According to WHO (World Health Organisation) charts and ESH (European Society of Hypertension) charts, the optimal blood pressure is lower—in both values, than 120/80 mmHg. The values between this border and the blood pressure of 139/89 mmHg are considered as prehypertension. The higher values on the other hand are considered as different stages of hypertensions.

TABLE 1
Categories of blood pressure (source WHO/ESH)

| Categories | systolic BP [mmHg] | diastolic BP [mmHg] |
|---|---|---|
| Optimal BP | <120 | <80 |
| Normal BP | <130 | <85 |
| Prehypertension | 130-139 | 85-89 |
| Hypertension | | |
| Modest | 140-159 | 90-99 |
| Border | 140-149 | 90-94 |
| Medium serious | 160-179 | 100-109 |
| Serious | ≥180 | ≥110 |
| Isolated systolic | ≥140 | <90 |
| Border systolic | 140-149 | <90 |

It is obvious that measuring the blood pressure is more than needful. With this is connected a new approach to control and monitoring of human health E-Health, whose aim is not only to monitor for example post-operation condition, individuals with health risks, but at the end it would be appropriate to monitor the whole population to prevent critical situations. This monitoring, methods usually connected with the complex of other measurements—temperature, heartbeat, breathing, oxygen content in blood etc., require finding such a method that burdens and obtrudes the subject as little as possible. These units for reading quantities can by either autonomous or connected with the assessment center and its assessment. Reading of quantities itself can be either continual or one-time, or both, usually is motivated from the measured subject. It is thus optimal if the device is a part and/or is connected to a modern mobile terminal such as SmartPhone.

Up to date solutions for determining the values of systolic and diastolic blood pressure are based on principles that can be divided into two categories, these are invasive and non-invasive methods.

The invasive method means inserting the reading component directly into the measured bloodstream and it is not relevant for the aim of this solution.

The non-invasive method can be divided to discrete methods—auscultation method, oscillometric method, palpation method, infrasound method, ultrasound method etc. and continuous methods—the vascular unloading method, the method of arterial stiffness, method of sensing of the speed of the pulse wave, etc.

All the above mentioned methods use the cuff either for a wrist or for an arm and afterwards they assess the values depending on the status of inflating this cuff.

An important fact is that with an exception of invasive methods, which obtrude the subject though, the majority of measurements evince a relatively considerable mistake, especially as far as the relation to health condition and the age of a subject is concerned. Considering this it is therefore optimal to do the measurements frequently and assess the changes, or eventually undertake a calibration. As a matter of fact it requires such a principal of measuring systolic and diastolic blood pressure that obtrudes the subject as less as possible or not at all, and all the procedures with the inflating cuff are therefore inconvenient.

The most used method out of the separate methods is the oscillometric method which is based on an assessment of oscillometric pulsations—pressure pulsations that are generated in the inflating cuff during its inflation and deflation. The crucial problem of this method is the still unclear criteria for assessing systolic and diastolic pressure. These values are usually determined by an application of the mathematic criteria on the envelope of oscillometric pulsations. Each manufacturer uses his own secret algorithms and therefore it is impossible to objectively find out sufficient accuracy and repeatability of measurements. Considering the energetic requirements and especially practical application, it is logical, that the measuring by means of inflatable cuff is not possible for continual monitoring of the subject. The same is true about other cited methods.

Another crucial problem of the currently used methods applying inflating cuffs is that older individuals and individuals with damaged blood stream, e.g. diabetes, where the bloodstream tends to return slowly to rest regime and the measurement is therefore not repeatable.

Usually it is not possible to inflate the cuff during the usual activity—it is not possible because of the technological, psychological and social point of view. The solution thus has to be non-invasive und unobtrusive.

To control the continuous but also one-time life functions—e.g. the heartbeat, temperature, breathing, the amount of oxygen in blood, systolic and diastolic pressure, if needed other quantities like ECG, EEG etc., the sets of specialized apparatus are used that only lately began to feature wireless interfaces enabling a connection to other devices such as personal computers, tablet and/or the terminal such as SmartPhone and/or databases and analytical programs.

There are many defined and standardized protocols for communication, that are possible to use for communication and transfer of measured data from the sensor network to the internal computer unit or for the transfer of the data to a remote network. It is namely Bluetooth (IEEE 802.15.1), for the communication at close distance, further GSM/HSCSD/GPRS, UMTS, LTE, WiFi (IEEE 802.11). At the same time there are in common usage systems GNSS-GPS, GLONASS, GALILEO, to determine location and time. There is a preference for the communication module of the type of Bluetooth (IEE 802.15.1) which is characterized by an autonomous control of its surrounding with a corresponding reaction to a call for communication when it switches from a standby mode with a very low consumption to a mode of communication and after the exchange of the data it returns back to a standby mode.

The sensor units are crucial for monitoring which enable contactless collection of data and their transfer to superordinate facilities.

However, the current apparatus for monitoring and measuring the human life functions use only some of the above mentioned standards and that only separately. They represent therefore separate, relatively large-sized units utilized mostly to a measurement of one quantity. The first apparatus that combine more functions appear either in the field of satellite navigation, where they together with another measured quantity, usually heartbeat, serve as a tool for sport activity and/or are implemented as single-purpose ones to monitor one of the life functions' parameters. Typical examples are units for measuring the content of oxygen in blood. In these cases they are usually solved as a cuff and/or finger-mounted module or earlobe-mounted module and the collected data are subsequently transported either by overcrossing wiring and/or wirelessly to the next unit. These units usually feature their own display and operating components. Their main disadvantage is that not only they are not utilized to long-term measurement, but also their high energy consumption demands large-sized power supplies and doesn't allow long-term measurements—in this case it is basically a question of the future technology though. The second main disadvantage is that units are not adjusted for long-term wearing and therefore are not practical as far as any activity other than measuring is concerned.

### DISCLOSURE OF INVENTION

The above mentioned drawbacks are eliminated by the method of determining systolic and diastolic blood pressure from parameters of plethysmographic curve without the necessity of applying the inflating/deflating cuff supplemented with the technical device for monitoring and measuring human life functions that utilizes the telecommunication technology with electronics and sensors, computer technology and the wireless communication, factually the solution concerns the unit for monitoring and measuring human life functions by a specifically adapted electronic module and its communication with other devices.

The default point is the plethysmographic curve. The plethysmographic curve is obtained on the basis of passing through a tissue, so called transmission way of rays of light, while using the reflective method it is possible to work with the reflected light energy. Usually the radiance in the region of the infrared light is used. The device usually houses the light source, scanning element, for example photodiode, phototransistor, camera, etc. and an assessment that can display the transmitted information and/or transfer it for further processing. The plethysmographic curve can be obtained also as another product while measuring the oxygen content in blood. Commonly radiation through a tissue by two different rays of light, usually in the red and infrared regions of spectrum is used.

The plethysmographic curve is digitized and undergoes the filtering and segmentation with the consequent choice of valid segments, and the values of systolic and diastolic blood pressure of the measured subject are determined from the valid segments.

The principle of the invention is that continuously or one-time obtained plethysmographic curve is processed by the procedure according to the invention and resulting values are proclaimed as systolic and diastolic blood pressure of the measured subject—a human.

If the systolic and diastolic blood pressure is determined in a way described according to this invention, the set of obtained data of the plethysmographic curve is divided into separate segments that respond to one heartbeat of the measured subject and they represent one cycle of the heart activity where each segment is defined as two local minimums determined by the beginning and the end of one global maximum between them, with the advantage of 20 segments. In order to eliminate the saturated signal from further evaluation, the minimum and maximum values of the main maximum in relation to saturation of the signal determining the constant Kₐ and its minimal value K_{aMIN} and maximal value K_{aMAX} and the saturated parts of the plethysmographic curve are excluded from further assessment. The constant Kₐ is with the advantage 10 for minimal value K_{aMIN} and with the value 95% of the total maximum in values according to the type A/D converter of the transfer for maximal value K_{aMAX}. The course of the left segments of each section of the plethysmographic curve is filtered by calculating means of subsequent values of the plethysmographic curve. The number of segments used for calculation of the mean is related to the used monitoring element in such a way that no significant distortion of the final shape arises and it defines the constant Kₚᵣᵤₘ, and in each section the minimal and maximal value is determined. The maximal value in each section is matched with the value 1, whereas the minimal value is matched with the value 0. All other values of the pletysmographic curve, with respect to each section, are linearly transformed into a dynamic range 0 to 1. In each section the set of the plethysmographic curve data is divided into individual segments which correspond to one heartbeat of the measured subject and represent one cycle of the heart activity where each segment is defined as two local minimums set by the beginning and the end and one global maximum between them, with the advantage up to 20 segments.

Segments, one independently on the other, are normed as follows: the value Y of the global maximum is matched with the value 1 and the value 0 is determined as a result of the arithmetic mean of the beginning and the end of the segment for the segments with the difference lower than 5% and the samples are linearly transformed according to those limits and/or the values of the plethysmographic curve for segments with the difference lower than 25% are recalculated so that the beginning and the end of each segment has the same value 0 and this defines the shift in the offset value and the multiplicative constant gain and chosen segments are again linearly transformed using values offset and gain and the area of each chosen segment is divided into 6 parts defined as dividing vertical from the maximum and divided in the axis Y for values between 20 to 60% and 40 to 90% out of maximum, where the second dividing must be percentage larger than the first one. In chosen segments the steepness of systolic run-up is determined as the first derivation of an edge of each chosen segment. Then areas and centers of gravity of all 6 parts are calculated and further closed areas and/or a line are created from the points of centers of gravity. Further total areas and centers of gravity for the selected segment are determined as well as the area and the center of gravity of the new area created from centers of gravity of segment partial areas and/or the size of the line determined by partial points and/or the same in combination with the center of gravity of the whole plethysmographic curve. At the same time the constant K_{ind} is determined as the value of the statistic mean of samples of checking measurements of subjects for systolic and diastolic blood pressure and the constant Kₙ as the value of the statistic mean of samples of checking measurements for systolic and diastolic blood pressure of the subjects, divided according to age and gender categories, and identically the constant K_{R} is determined in the same manner as the constant of the basic range segmented for systolic and diastolic pressure, and the systolic pressure is determined as a product of

K_{RS}*K_{nS}*K_{inDS}*D*S8

or

K_{RS}*K_{nS}*K_{inDS}*D*ST67810

or

K_{RS}*K_{nS}*K_{inDS}*D*(TyST67810*TxST67810)

furthermore diastolic pressure as the product of

K_{RD}*K_{nD}*K_{indD}*((U911)*S)

or

K_{RD}*K_{nD}*K_{indD}*(√((Tx9-Tx1)²+(Ty9-Ty11)²)*S)

together with determining the diastolic pressure as the product of

K_{RD}*K_{nD}*K_{inD}*((TyST*TxST)*S)

or

K_{RD}*K_{nD}*K_{indD}*STT

or

K_{RD}*K_{nD}*K_{inD}*(TySTT*TxSTT)

The segment must be longer than minimal and shorter than maximal allowed limit, while limits are defined in time constants equivalent to number of assessed pulses of heartbeat starting with 5 pulses as the minimum up to 250 pulses as the maximum.

Determining the number of samples used for calculating of the mean is defined advantageously as the constant Kₚᵣᵤₘ, this constant is determined according to the type of scanning unit in relation to actual disturbance of its amplifier and it represents a compromise in a sense that it smoothes desirably a signal and it still does not undesirably and considerably distort the signal, especially its rapid changes in the vicinity of local extremes. The value of the constant Kₚᵣᵤₘ is with the advantage in the range 2 to 30 in terms of consecutive samples.

The constant K_{R} is the constant of the basic range of systolic pressure and is with an advantage determined as K_{RS} for systolic pressure with the basic value K_{aMIN}/10 and it is with an advantage determined as K_{RD} for diastolic blood pressure with the basic value K_{aMIN}/100.

The division into partial areas 6, 7, 8, 9, 10, 11 is with an advantage determined as 35% and 55% out of maximum.

The constant K_{ind} equals implicitly 1 for systolic and diastolic blood pressure.

The significant advantage according to the invention is that scanning the plethysmographic curve is entirely non-invasive, without any sound effects, energetic requirements are lower than typical compressor and its electromotor. The significant advantage also is that the device does not include any movable parts. Its usage is in terms of health harmless and can be applied for arbitrarily long period. There is a reasonable assumption that especially with the older subjects and subjects with damaged blood stream, the final determining of systolic and diastolic blood pressure is more accurate and the resulting measurements are repeatable as they don't influence the status of the blood stream in any way.

The above mentioned drawbacks are considerably eliminated by the unit for monitoring and measuring human life functions, according to this invention. It is based on a carrier construction in a shape of a ring and/or an earring equipped with a transmitting sensor, another transmitting sensor, scanning sensor for transmitting sensor, other transmitting sensor and scanning sensor for measuring temperature of surrounding, that are connected via interface to a communication module unit wirelessly connected to another communication module placed out of the ring and/or the earing and interconnected to a supervising controlling unit, at the same time the transmitting sensor, another transmitting sensor and other transmitting sensor, scanning sensor for measuring temperature of surrounding and the communication module unit are powered from battery implemented in the ring which is placed on the fingertip and/or the earlobe of the monitored and measured subject.

The transmitting sensor can be furnished with an infrared radiation supply for the plethysmographic curve data supply of the measured subject scanned by the scanning sensor and/or infrared radiation supply for the plethysmographic curve supply scanned by the scanning sensor.

The networks made of sensors are connected to each other directly to the communication module via its interface and then wirelessly, using the network of short coverage, the data are transferred to another device, all the electronics is integrated in a shape of the ring and/or the earring and it is adapted for long-term measurements and wearing, at the same time, the data assessments as well as the other calculations are centralized into a superordinate unit, which is of any type, but has its communication unit.

The invention is based on a communication module unit connected via interface for communication module connection to both, the transmitting sensor and to another transmitting sensor at the same time and further it is attached to the scanning sensor and another scanning sensor for measuring temperature of the ring surrounding and/or the surrounding of the earlobe inside the earring, all the modules are supplied from the inbuilt battery and they are places inside the mechanical construction that forms the ring and/or the earing put on a finger and/or an earlobe of the monitored and measured subject—a human, the modules wirelessly transmit and receive data by means of the communication module to the superior controlling, assessing and computing unit.

The main construction advantage is the simplicity of electronic blocks that at the end enables a simple processing of the construction of the ring and/or the earring itself and its design, the design can even follow the fashion jewellery. The construction of installation of the scanners in a shape of the ring and/or the earring ensures reliable transit transition of measured signals and their assessment from the point of view of measured human tissue. As a consequence these are not that critical movements of measured subject in the course of the measurement and the measurement itself is more stable and accurate.

### EXPLANATION OF FIGURES IN THE DRAWINGS

The invention will be described in more detail in particular examples and described in enclosed drawings, where

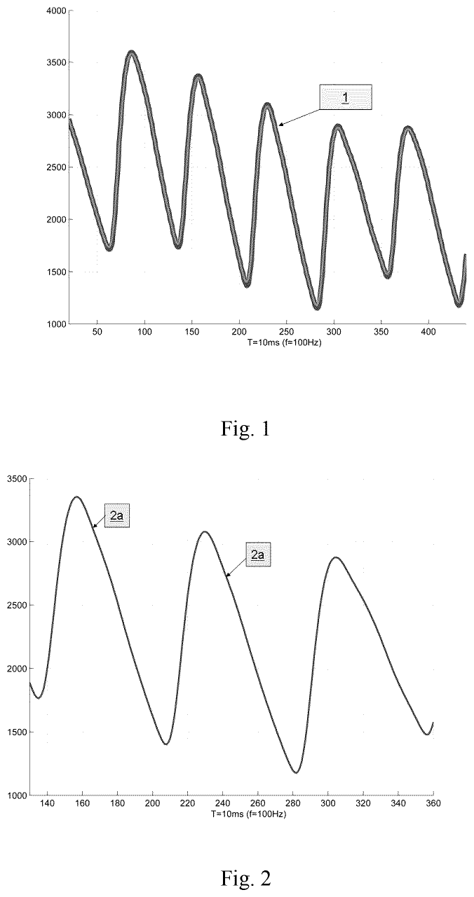

the FIG. 1 shows the plethysmographic curve—the example of the data set.

The FIG. 2 shows the segments in the course of the plethysmographic curve.

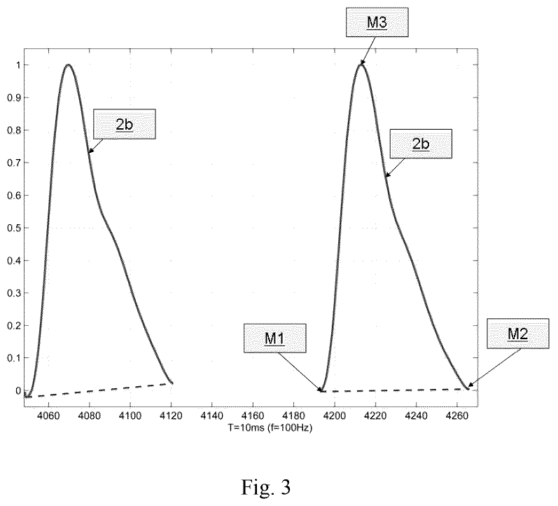

The FIG. 3 is an example of two valid normalized selected segments of the plethysmographic curve.

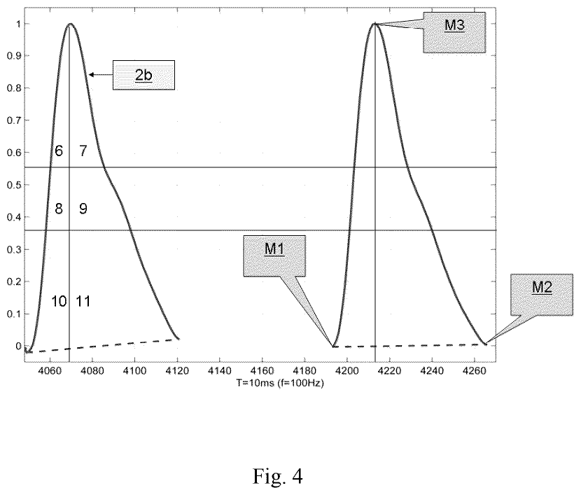

The FIG. 4 is an example of two valid normalized and selected segments of plethysmographic curve and its division into partial segments.

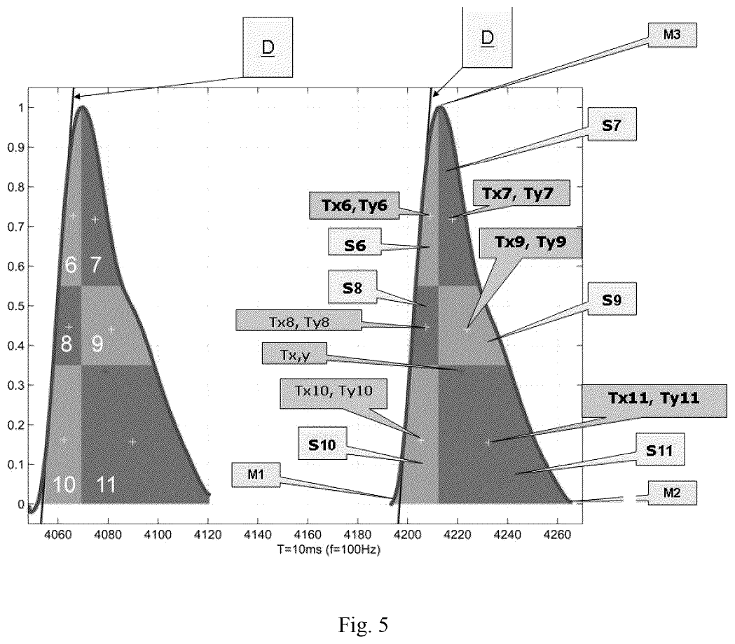

The FIG. 5 is an example of two valid normalized segments of the plethysmographic curve in the layout of connected lines M1, M2, M3 and its division of the partial segments.

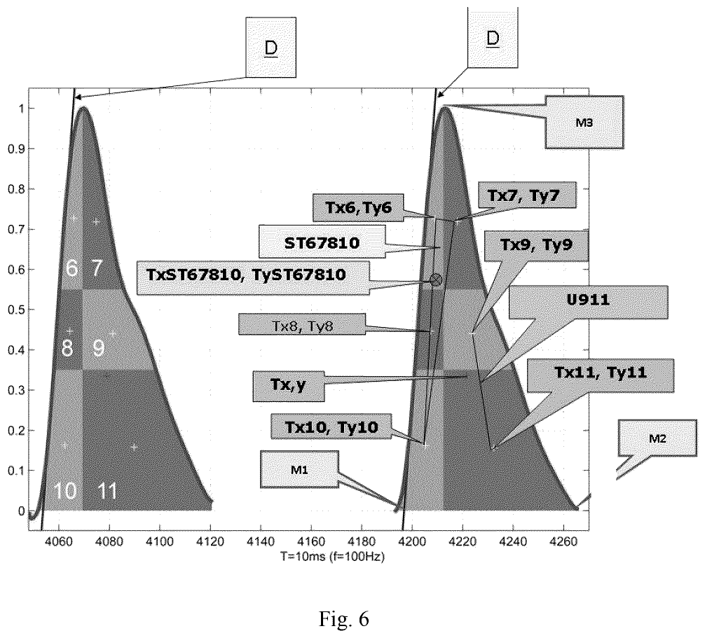

The FIG. 6 states the features evaluated from the plethysmographic curve and

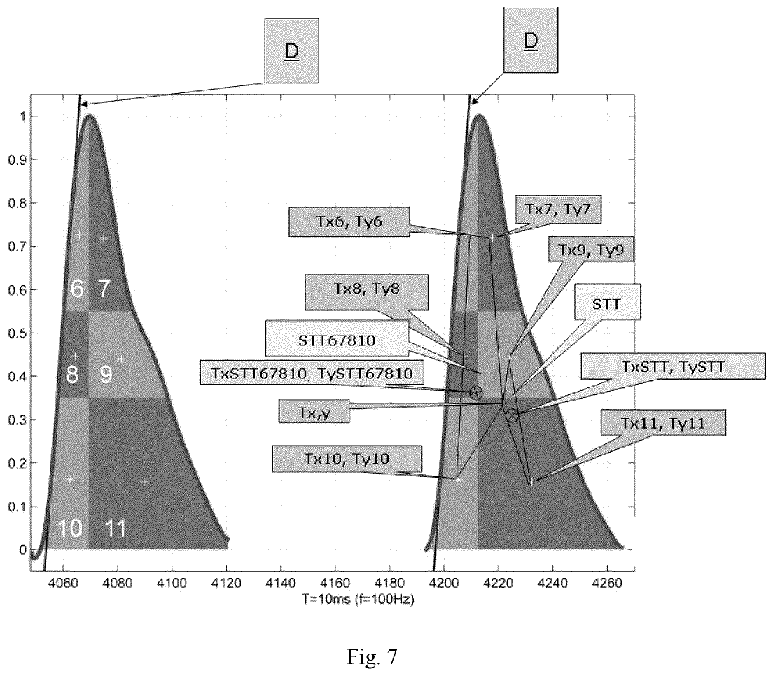

the FIG. 7 shows the features evaluated from the plethysmographic using new areas determined from centers of individual segments and the total center. The technical solution is explained in more detail in the enclosed drawing, where in

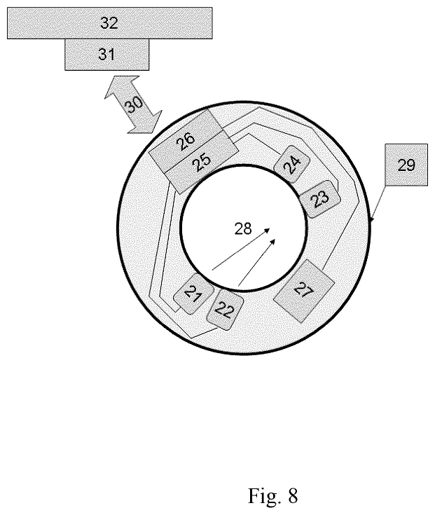

the FIG. 8 for the ring construction and in

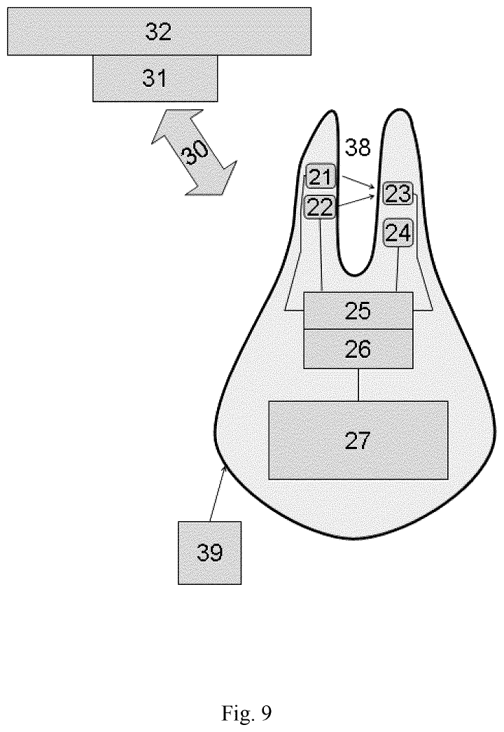

the FIG. 9 for the earring construction, there is an overview scheme of solution and communication and it also shows the block scheme of a sensor network and communication modules.

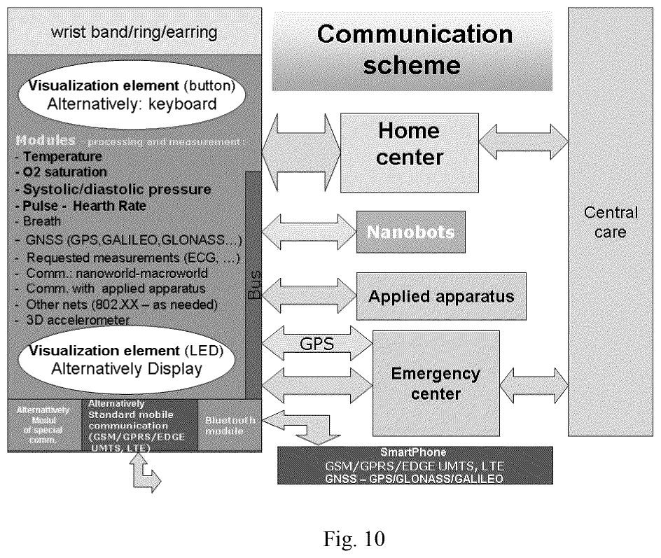

The FIG. 10 shows an example of communication of the unit that provides the systolic and diastolic blood pressure determination in cooperation with the surrounding environment.

### EXAMPLES OF INVENTION IMPLEMENTATION

An example of the way of determining systolic and diastolic blood pressure according to the invention is described in a particular example of processing—FIG. 1 to 7.

The set of data obtained from separate curves of plethysmographic curves 1 are split into segments 2a where one segment 2a corresponds to one heartbeat of the measured subject and it represents one cycle of the heart activity, it is therefore a section of examples of the plethysmographic curve 1 between two local minimums M1 and M2 while segment a includes one global maximum M3 between the beginning and the end of the segment 2a, the segment 2a must be longer than the minimal and shorter than the maximal allowed limit, these are defined in constants of time units that are equivalent to a number of evaluated pulses of the heart beat and this course of segments 2a of the plethysmographic curve 1 is divided into individual sections containing one or more segments 2a with the advantage of division to sections up to 20 segments 2a. To exclude the saturated signal from further assessment, the saturated parts of segments 2a of the plethysmographic curve 1 are excluded from further assessment according to the constant Kₐ and the left segments 2a are filtered by calculating the mean of subsequent curve sample values according the constant Kₚᵣᵤₘ. Each segment 2a, one each independent on the other, is checked from the point of view of the pertinence of measured data in the manner that the differences of values of the Y coordinate of the beginning M1 and the end M2 of the curve are checked and only left selected segments 2b are standardized, one each independent on the other in a way, that Y value of the global maximum 3 is assigned with the value 1 and after the 0 value is determined as a result of the arithmetic mean of the beginning M1 and the end M2 of the selected segment 2b afterward the samples are linearly transformed considering these values obtained this way and/or the values for the pletysmographic curve 1 for segments a with the difference between the beginning M1 and the end M12 lower than 25% recalculate in a manner, that the beginning and the end of each segment has the same value and selected segments 2 are again linearly transformed, when the features for parameters of the curve such as the steepness of D of the systolic run-up as the first derivation of the leading edge of each selected segment 2b and the partial areas of each selected segment 2b are determined, the area of each selected segment 2b is divided into 6 parts marked 6, 7, 8, 9, 10, 11 and obtained as the dividing vertical from the maximum M3 of the selected segment 2b divided in the axis Y for the values 35% and 55% out of the maximum M3 and/or the area of each selected segment 2b is divided into 6 parts defined as the dividing vertical from the maximum M3 divided in the axis Y for the values between 20 to 60% and 40 to 90% out of the maximal value, where the second dividing line must be by percentage bigger than the first one. Now the total area S is determined for each selected segment 2b and identically the area of each part is determined, which is S6, S7, S8, S9, S10, S11 together with the center of gravity of each part as well as the whole segment, which means Tx, TY, Tx6, Tx7, Ty7, Tx8, Ty8, Tx9, Ty9, Tx10, Ty10, Tx11, Ty11 and then for each selected segment 2b the area ST67810 is determined using the centers of gravity of parts 6, 7, 8, 10 and/or the centers of gravity of parts 9, 11 that help to create the line U911 (Ux911, Uy911), and by means of the total center of gravity Tx,y of the plethysmographic curve 1 the other shapes with the area STT67810 and its centers of gravity TxSTT67810, TySTT67810 and the area STT and its centers of gravity TxSTT, TySTT are created. The constant Kind is determined as the value of statistic mean calculated from a large number of subjects' checking measurements' samples for systolic and diastolic pressure, implicitly it equals 1 and then the constant K_{ind} is determined from the set of samples of checking statistic measurements, the constant K_{ind} is divided into a part of the systolic pressure Kinds and the part of the diastolic pressure together with the constant Kₙ determined as the value of statistic mean of the large number of checking measurement's samples divided according to the age and gender category and the condition of the subject's blood stream divided in the part of the systolic blood pressure K_{indS} and diastolic blood pressure K_{indD} and the constant K_{RS} is determined form the basic range of the systolic pressure and the constant K_{RD} of the basic range of the diastolic range, afterwards the systolic blood pressure is determined as a result of

K_{RS}*Kₙₛ*K_{indS}*D*S8

and at the same time the diastolic blood pressure is determined as a result of

K_{RD}*K_{nD}*K_{indD}*((Ty*Tx)*S)

where the systolic blood pressure is determined as a result of

K_{RS}*K_{nS}*K_{indS}*D*ST67810

or

K_{RS}*K_{nS}*K_{indS}*D*(TyST67810*TxST67810)

and at the same time the diastolic blood pressure is determined as a result of

K_{RD}*K_{nD}*K_{indD}*((U911)*S)

or

K_{RD}*K_{nD}*K_{indD}*(√((Tx9-Tx11)²+(Ty9-Ty11)2)*S)

together with determining the diastolic blood pressure as a result of

K_{RD}*K_{nD}*K_{indD}*((TyST*TxST)*S)

or

K_{RD}*K_{nD}*K_{indD}*STT

or

K_{RD}*K_{nD}*K_{indD}*(TySTT*TxSTT)

where scanning of the plethysmographic curve itself is recommended to be done while resting, without any major movement of the measured subject and then according to a need, at the same time other determined parameters such as the total area S or the area of each part 6 to 11 marked S6, S7, S8, S9, S10, S11 and the center of gravity of each part as well as of the total segment Tx, Ty, Tx6, Ty6, Tx7, Ty7, Tx8, Ty8, Tx9, Ty9, Tx10, Ty10, Tx11, Ty11 are applied, as well as the area ST67810 and the centers of gravity TxST67810, TySTT6810 of the new area created from the centers of gravity of the segments' partial areas, respectively the size of the line determined by centers of gravity of partial points marked Ux911, Uy911, and/or the same in combination with the total center of gravity, labelled as the area STT67810 and centers of gravity TxSTT67810, TySTT67810 and the area STT and its centers of gravity TxSTT, TxSTT in an application for correction of the method of determining the systolic and diastolic blood pressure.

Determining of systolic blood pressure and diastolic blood pressure themselves is taken over from the source—either a separate device comprising the light source irradiating a tissue of the subject and the scanner scanning the final plethysmographic curve, or as the case might be from one channel forming the unit for oxygen content measuring and/or other similar device, the data corresponding to the tissue condition as described in the FIG. 1 and we proceed as follows:
1. The set of received data of each curve—FIG. 1 plethysmographic curves 1 consists of FIG. 2 segments 2a, one segment 2a corresponds to one heartbeat of the measured subject—a human and it represents one cycle of the heart activity. Segment 2a is therefore a section of plethysmographic curve examples between two local minimums M1 and M2 and segment 2a includes one global maximum M3 between the beginning and the end of the segment 2a—FIG. 3. Segment 2a must be longer than the minimal and longer than the maximal allowed limit. These limits are defined in constants of time units that are equal to number of evaluated pulses of heartbeat, e.g. 5 pulses as the minimum and 250 pulses as the maximum.
2. The course of segments 2a of the plethysmographic curve 1 is divided into individual sections containing one or more segments 2a with the advantage of dividing into section up to 20 segments 2a and to exclude the saturated signal from further assessment the other saturated parts of segments 2a of the plethysmographic curve 1 are then in accordance with the constant Kₐ excluded from further processing. Recommended values are always determined according to the type of the scanning unit as the constant Kₐ or its limits, which means K_{aMin} and K_{aMAX}. See an example in FIG. 1 or FIG. 2, where the recommended value will be K_{aMIN}=1000 for the minimal value and K_{aMAX}=4995 for the maximal value. Both values enable exclusion of the saturated segments. The constant K a advantageously equals 10 for the minimal value K_{aMIN} and with the value that equals 95% out of the total maximum in values according to the type A/D of the transfer for the maximal value K_{aMAX}.
3. The course of segments 2a of the plethysmographic curve is filtered by making an average from the subsequent values of the curve examples. The determination of segment number used for averaging is defined as the constant Kₚᵣᵤₘ and is defined according to a type of the scanning unit, in connection to its own interference of its amplifier and it is a compromise in a sense that the signal is desirably purified and at the same time it still doesn't affect the signal significantly, so the rapid changes near the local extremes are not distorted—FIG. 1 and FIG. 2. The typical value Kₚᵣᵤₘ ranges for contemporary amplifiers from 2 to 30 subsequent samples.
4. In each section the maximal and minimal values are determined. In each section the maximal value is matched with the value 1 and the minimal value is matched with the value 0. All other values of the plethysmographic curve, with respect to each section, are linearly transformed into a dynamic range 0 to 1.
5. Each segment 2a, one independently on another, is checked from the point of view of its relevance of measured data. Therefore the differences of value Y and coordinates of the beginning M1 and the end M2 of the curve are checked. Only the selected segments 2b that differ by a defined quantity—e.g. 5% are left and/or the values of the plethysmographic curve (1) are recalculated in a way that the beginning and the end of each segment's minimum have the same value for differences lower than 25%. This value corresponds to the final accuracy of a measurement of the resulting pressure. See FIG. 2 and FIG. 3.
6. Left selected segments 2b are nonmed, one independently on another in that way, that the Y value of the global maximum M3 matches the value 1. See FIG. 3.
7. The value 0 is determined as a result of the arithmetic average of the beginning M1 and the M2 of the segment for segments with the difference lower than 5%, and/or the values of the plethysmographic curve recalculated in the way, that that the beginning and the end of each segment's minimum have the same value. This defines the shift in value (offset) and the multiplicative constant (gain).
8. Examples are linearly transformed by values offset and gain (FIG. 3).
9. Features for curve parameters are determined in selected segments 2b, namely
   the steepness of the systolic run-up is determined as the first derivation of the leading edge of each selected segment 2b—quantity D
   partial areas of each selected segment 2b described in FIG. 4, FIG. 5, FIG. 6 and FIG. 7 are determined by dividing the area of each selected segment 2b into 6 parts defined by a dividing vertical from the maximum M3 of the segment divided in the axes Y for values 35% and 55% out of maximum M3.
   partial areas of each segment are numbered according to the FIG. 4, FIG. 5, FIG. 6 and FIG. 7 respectively.
10. For each selected segment 2b area parameters are determined:
   total area S
   area of each part 6 to 11 is numbered S6, S7, S8, S9, S10, S11
   the center of gravity of each part and of the whole segment, namely Tx, Ty, Tx6, Ty6, Tx7, Ty7, Tx8, Ty8, Tx9, Tx10, Ty10, Tx11, Ty11
11. For each selected segment 2b its parameters of areas and centers of gravity are determined using individual centers of gravity of parts 6, 7, 8, 10:
   the total area ST67810 of the inner center of gravity
   the centre of gravity TxST67810, TyST67810 of this segment.
12. For each selected segment 2b its area parameters are determined using the individual centers of gravity of parts 9,11:
   line U911
13. For each selected segment 2b its area parameters are determined using individual centers of gravity of parts 6,7,8,10 and the total center of gravity Tx,y of the plethysmographic curve:
   total area STT67810 of the inner center of gravity
   centers of gravity TxSTT67810, TySTT67810 of this segment.
14. For each selected segment 2b the area parameters are determined using its individual centers of gravity of parts 9,11 and the total center of gravity Tx,y:
   total area STT of the inner center of gravity
   centers of gravity TxSTT, TySTT of this segment.
15. The constant K_{ind} is determined as the value of the statistic average of the large number of subject checking measurement examples for systolic and diastolic blood pressure, implicitly it is 1. Its actual value that serves as a correction of value referring to a particular subject can be determined as a result of comparison on checking measurement, laboratory measurement of the subject in comparison with the value stated according to paragraphs 17 to 20. This measurement is carried out only in necessary cases when there is a request for the highest possible accuracy of the resulting determination of systolic and diastolic pressure. The constant K_{ind} is in this case divided into a part of the systolic blood pressure K_{indS} and diastolic blood pressure K_{indD}. A standard accuracy of measurement without any individual correction K_{ind} must be better than ±15%. It is appropriate to make the measurements at rest, without a move of a subject according to recommended control in accordance with the paragraph 21. The constant K_{ind} is used as a correction for subjects that have vast damages on blood stream that are typical for some diseases such as diabetes etc. It concerns the cases when the standard classification of a subject into one of categories of the constant Kₙ according to the paragraph 16 is not sufficient.
16. The constant Kₙ is determined as the value of the statistic average of a large number of subject checking measurement examples divided according to the gender and age category. The constant shows the status of the subject's blood stream in dependence on gender, age, its diseases as the case might be, e.g. diabetes, atherosclerosis etc. The constant Kₙ is split into a part for the systolic blood pressure K_{nS} and the part for the diastolic blood pressure K_{nD} and that in all following categories of subject classification.

The statistic value is controlled in three basic categories:
juniors for the age category 10-20 years . . . Kⱼᵤₙ meaning K_{junS} and K_{junD}
adults for the age category 21-50 years . . . Kₛₜ meaning K_{stS} and K_{stS} and K_{stD}
seniors for the age category 50 years and over . . . Kₛₑₙ meaning K_{senS} and K_{senD}

Its value can be affected by the measuring channel that provides the plethysmographic curve, but only as a multiplicative constant.
17. The systolic blood pressure is determined as a result of

K_{RS}*K_{nS}*K_{indS}*D*S8
where K_{nS}=K_{inS} or K_{sjS} or K_{senS} according to a type of the measured subject and his/her age
   K_{ind}=K_{indS} in cases specified in the paragraph 15, if the implicit value 1 is not sufficient
   D is the steepness of the systolic run-up defined as the first derivation of the run-up of each selected segment 2b of the plethysmographic curve 1
   K_{RS} is the constant of the basic range of the systolic blood pressure; with advantage it is possible to use the initial value K_{aMIN}/10
18. The diastolic blood pressure is determined as a result of

K_{RD}*K_{nD}*K_{indD}*((Ty*Tx)*S)
where K_{nD}=K_{junD} or K_{stD} or K_{senD} according to a type of the measured subject and its age
   K_{ind}=KindD in case described in the paragraph 15, if the implicit value 1 is not sufficient
   K_{RD} is the constant of the basic range, it is possible to use it advantageously as a default value K_{aMIN}/100
19. The systolic blood pressure is determined as a result of

K_{RS}*K_{nS}*K_{indS}*D*ST67810

and/or

K_{RS}*K_{nS}*K_{indS}*D*(TyST67810*TxST67810)
where K_{nS}=K_{junS} or K_{stS} or K_{senS} according to a type of the measured subject and its age
   K_{ind}=K_{indS} in case described in the paragraph 15, if the implicit value 1 is not sufficient
   D is the steepness of the systolic run-up which is determined as the first derivation of the leading edge of each selected segment 213 of the plethysmographic curve 1
   K R s is the constant of the basic range of the systolic pressure, it is possible to use it advantageously as a default value K_{aMIN}/10) 20. The diastolic blood pressure is determined as a result of

K_{RD}*K_{nD}*K_{indD}*((U911)*S)

specifically

K_{RD}*K_{nD}*K_{indD}*(√((Tx9-Tx1)²+(Ty9-Ty11)²)*S)

and/or

K_{RD}*K_{nD}*K_{indD}*STT

and/or

K_{RD}*K_{nD}*K_{indD}*(TySTT*TxSTT)
where K_{nD}=K_{junD} or K_{stD} or K_{senD} according to the type of the measured subject and his/her age
   K_{ind}=K_{indD} in cases specified in the paragraph 15, if the implicit value 1 is not sufficient
   K_{RD} is the constant of the basic range; beneficially it is possible to use the initial value K_{aMIN}/100
21. The acquisition of the plethysmographic curve 1 is recommended to be carried out at rest, without a significant movement of the measured subject. The acquisition is recommended in relation to e.g. a 3D movement sensor with determining its limits
22. Other calculated parameters, specifically:
   total area S
   area of each part 6 to 11 numbered S6, S7, S8, S9, S10, S11
   center of each part and of the whole segment, namely Tx, Ty, Tx6, Ty6, Tx7, Ty2, Tx8, Ty8, Tx9, Tv2, Tx10, Ty10, Tx11, Ty11
   total area ST67810 created using the centers of gravity of parts 6, 7, 8, 10
   the centers of gravity TxST67810, TyST67810 of this area, line U911 created from centers of gravity of parts 9, 11
   the area STT67810 created using the centers of gravity of parts 6, 7, 8, 10,
and the center of gravity Tx,y,
   the center of gravity TxSTT67810, TySTT67810 of this area
   the area STT67810 created using the centers of parts 9, 11 and the center Tx,y, so
   the centers of gravity TxSTT67810, TySTT67810 of this area are possible for correction of the manner of calculation according to paragraphs 17 to 20 for specific cases of further more accurate value determination of systolic and diastolic blood pressure on the basis of checking measurement of large number of subjects that have specific features of their blood stream.

FIG. 1 shows an example of the plethysmographic curve 1 taken by the input module and transferred for pressure determination. The curve shows the quality of blood supply in tissues and provides information about the reactivity of the blood circulation. The plethysmography makes it possible to get the record of pulse waves with the help of a sensor and a source of light.

FIG. 2 shows how the plethysmographic curve 1 is divided into segments matching one heartbeat.

FIG. 3 shows how the valid segments of the plethysmographic curve 1 are made depending on value quantity of points M1 and M2.

FIG. 4 shows the split of valid segments into partial segments by determination of the dividing borders in the axis Y.

FIG. 5 shows two valid normalized segments of the plethysmographic curve 1, their labelling of separation into partial segments and determination of areas and centers of gravity.

FIG. 6 shows features of the plethysmographic curve 1 where the new areas and a line determined from each segment's centers are used.

FIG. 7 shows features evaluated from the plethysmographic curve 1, where the new areas determined from each segment's centers and the total center are used.

An example of the layout of the unit for monitoring and measuring human life functions according to the invention is introduced as a block in the attached drawings in FIG. 8 and FIG. 9, once for the constructional layout in a shape of the ring—FIG. 8 and once for the constructional layout in a shape of the earring—FIG. 9. FIG. 10 is an example of the communication of the unit necessary to determine systolic and diastolic blood pressure in a cooperation with the surrounding environment.

The unit 26 of the communication module is connected over the interface 25 for communication module connection to both, the transmitting sensor 21 and another transmitting sensor 22 and at the same time it is connected to the scanning sensor 23 and also to the scanning sensor 24 for surrounding temperature scanning, while all the modules are supplied from the inbuilt battery 27 and are placed in the space of the mechanical construction that forms a ring 29 and/or an earring 39 put on the finger 28 and/or the earlobe 38 of the monitored and measured subject—a human, and using the wireless communication 30 they transfer and receive data by means of another communication module 31 to the superordinate unit 32.

During measurement, the assessment and controlling 32 gives an order by which another communication module 31 sets into operation and by the wireless communication 30 the communication module 26 is activated and over the interface 25 for the connection the scanning sensors 23 and 24 are controlled, these modules read the surrounding temperature of the finger 28 or the earlobe 38 as well as the value statuses transmitted by the transmitting sensor 21 and the other transmitting sensor 22 and transfer these data by the interface 25 and communication scanning module 26 and other communication module 31 to the controlling, assessment and computing unit 32.

By means of this unit 32 it is possible to monitor and measure continuously human life functions such as heartbeat, temperature, breathing, oxygen content in blood, systolic and diastolic blood pressure, or alternatively also other quantities such as ECG, EEG etc., and by wireless interface enables the connection to other devices as personal computer, tablet and/or terminal of the type Smart Phone and the like.

The programming equipment of the assessment and controlling unit 32 processes the data afterwards and displays them in a graphical or numeric shape, alternatively it transfers them to other superordinate units.

The obtained data can be further processed for example to determine oxygen content in blood, number of pulses of the heart activity and/or calculation of systolic/diastolic pressure.

### INDUSTRIAL APPLICABILITY

The way of systolic and diastolic blood pressure determination according to this invention will have a wide range of use. It will be employed for usual civil individual care, in the field of the medical care and postoperative monitoring of patient's condition, but especially it will serve as one of the foundations of the E-Health networks for future check-ups of all subjects and that way it will help to prevent critical health conditions.

The integrated system for collecting, processing and distribution of data, especially in the shape of the ring, where the sensor networks are connected by each other directly to the communication module over its interface and further wirelessly, using the short coverage network, are the data transferred to another device, where the set-up is adapted for long-term measuring and wearing, at the same time the complete controlling module and assessment of the data, together with other calculations, are concentrated to a superordinated unit of any type, only equipped with a communication module—controlling unit.

1-14. (canceled)

15. A method of determining systolic and diastolic blood pressure, wherein the received data set of separate curves of plethysmographic curve (1) is divided into individual segments (2a) with two local minimums given by the beginning (M1) and the end (M2) and one global maximum (M3) that matches one heartbeat of the measured subject and represents one cycle of the heart activity, the course of segments (2a) of the plethysmographic curve (1) is then divided into individual sections containing at least one segment (2a) with the division up to 20 segments (2a) and for the exclusion of the saturated signal from further assessment are according to the constant Kₐ the saturated parts of segments (2a) excluded from further assessment and the recommended values are always determined according to a type of the scanning unit as the constant Kₐ respectively its limits, specifically KaMIN a KaMAX, and the course of the plethysmographic curve (1) is then filtered by making averages of consecutive values of the plethysmographic curve (1), each segment (2a) is checked for differences between the value Y of the coordination of the beginning (M1) and the end (M2) of the curve and only selected segments (2b) are left, these segments up to 25%, selected segments (2b) are being normed so that the value Y of the global maximum (M3) is matched with the value 1 and the value 0 is determined as a result of the arithmetic average of the beginning (M1) and the end (M2) for selected segments (2b) with the difference lower than 5% and/or the values of the plethysmographic curve (1) for the selected segments (2b) with the difference lower than 25% are recalculated in the manner that the beginning and the end of the selected segment (2b) have the same value, and are divided into 6 parts defined as the dividing vertical out of the maximum (M3) divided in the axis Y for values between 20 to 60% and 40 to 90% out of the maximum (M3), where the second dividing must be by percentage larger than the first one and the partial areas (S6, S7, S8, S9, S10, S11) are determined as well as the centers of gravity (Tx6, Ty6, Tx7, Ty7, Tx8, Ty8, Tx9, Ty9, Tx10, Ty10, Tx11, Ty11) of each part and the centers of gravity (Tx, Ty) of the whole selected segment (2b), at the same time the constant (K_{ind}) is determined as the value of the statistic average of subject checking measurements example for systolic and diastolic pressure and the constant (Kn) as the value of the statistic average of the subject controlling measurements divided according to the age category and that together with a determination of the range constant (KR) of the measurement range, the quantity (D) is determined as the first derivation of the leading edge of each segment (2a) and now the systolic blood pressure is determined as a result of KRS*KnS*KindS*D*S8 and the diastolic blood pressure is determined as a result of KRD*KnD*KindD*((Ty*Tx)*S).

16. The method according to claim 15, comprising determining the partial areas (S6, S7, S8, S9, S10, S11) and the centers of gravity (Tx6, Ty6, Tx7, Ty7, Tx8, Ty8, Tx9, Ty9, Tx10, Ty10, Tx11, Ty11) of each part and centers of gravity (Tx, Ty) of the whole selected segment (2b) of the plethysmographic curve (1) and using these the new area (ST67810) is determined with its center of gravity (TxST67810, TyST67810) and/or the line (U911) and/or with the whole centers of gravity (Tx,Ty), areas (STT67810) and (STT) with their centers of gravity (TxSTT67810, TySTT67810) and (TxSTT, TySTT) and further the systolic blood pressure is determined as a product of KRS*KnS*KindS*D*ST67810 respectively KRS*KnS*KindS*D*(TyST67810*TxST67810) and at the same time the diastolic blood pressure is determined as a product of KRD*KnD*KindD*((U911)*S) which means KRD*KnD*KindD*(√(Tx9-Tx11)2+(Ty9-Ty11)2)*S), together with determining of the diastolic blood pressure as a result of KRD*KnD*KindD*((TyST*TxST)*S) and/or KRD*KnD*KindD*STT, respectively KRD*KnD*KindD*(TySTT*TxSTT).

17. The method according to claim 15, wherein the segment (2a) must be longer than minimal a and shorter than maximal allowed limit while the limits are defined in constants of the time units equal to a number of evaluated pulses of the heart beat from 5 pulses as the minimum to 250 pulses as the maximum.

18. The method according to claim 15, wherein the determining the number of selected segments (2b) used for averaging is defined as a constant (Kprum), it is determined according to a type of the scanning unit in connection to its own interference of its amplifier and it is a compromise in a sense that the signal is desirably purified and at the same time it still doesn't affect the signal significantly, so the rapid changes near the local extremes are not distorted.

19. The method according to claim 18, wherein the value of the constant (Kprum) is typically in the range from 2 to 30 of consecutive samples.

20. The method according to claim 15, wherein the matching the value Y of the global maximum (M3) with the value 1.

21. The method according to claim 15, wherein the determining the value 0 as a result of the arithmetic average of the beginning (M1) and the end (M2) of the selected segment (2b) which defines the shift in the offset value, the multiplicative constant gain is defined according to the claim 6 and the selected segments (2b) are one-dimensionally transformed with the help of values offset a gain.

22. The method according to claim 15, wherein the area of each selected segment (2b) that is divided into 6 parts defined as the dividing vertical form the maximum (M3) divided in the axis Y for values 35% and 55% out of maximum (M3).

23. The method according to claim 15, wherein the only selected segments (2b) are left that differ of 5% at maximum.

24. The method according to claim 15, wherein the values of the plethysmografic curve (1) for selected segments (2b) with the difference lower than 25% that are recalculated so the beginning and the end of minimums of each selected segment (2b) have the same value and the recalculated segments (2b) are transformed using the values offset and gain.

25. The method according to claim 15, wherein the area of each selected segment (2b) is divided into 6 parts defined as the dividing vertical out of the maximum (M3) divided in the axis Y for values 35% and 55% out of the maximum (M3), then areas and centers of gravity for all 6 parts are determined and from the points of centers of gravity new closed areas and/or lines are created and further there are determined total areas and centers of gravity for the selected segment (2b) as well as the area and the center of gravity of the new area created from centers of the partial areas of the segment and/or a size of the line determined from partial points of centers and/or the same in combination with the center of gravity of the whole plethysmographic curve (1).

26. The unit to operate the method according to claim 1, formed by the unit for monitoring and measuring the life functions of a human, wherein it comprises a supporting construction in a shape of a ring (29) and/or an earring (39) supplied with the transmitting sensor (21), other transmitting sensor (22), scanning sensor (23) for the transmitting sensor (21) and another transmitting sensor (22), and scanning sensor (24) for measuring the surrounding temperature, that are connected via interface (25) to the unit (26) of the communication module, which is by means of the wireless communication (30) connected to another communication module (31) placed outside the ring (29) and/or the earring (39) and connected to the superordinated controlling unit (32), while the transmitting sensor (21), another transmitting sensor (22), scanning sensor (23), scanning sensor (24) and the unit (26) of the communication module are powered from the battery (27) built-in the ring (29) and/or earring (39), which is attached to a finger (28) and/or to the earlobe (38) of the scanned and measured subject, where the transmitting sensor (21) is fitted with the radiation supply working in the infrared area of the spectrum and/or other transmitting sensor (22) is fitted with the radiation supply in the red range of the spectrum, for the supply of the plethysmografic curve (1) data of the measured subject scanned by the scanning sensor (23).

27. The unit according to the claim 26, wherein the transmitting sensor (21) that is equipped with the LED radiation supply working in the infrared range of the spectrum.

28. The unit according to the claim 26, wherein the transmitting sensor (22) that is equipped with the LED radiation supply working in the red range of the spectrum.

29. The unit according to claim 26, wherein the communication module (26) and/or another communication module (31) is formed by the module Bluetooth working according to the standard.
